(19) United States
(12) Patent Application Publication
(10) Pub. No.: US 2021/0260128 A1
(43) Pub. Date: Aug. 26, 2021

(54) NF-KB p50 DEFICIENT IMMATURE MYELOID CELLS AND THEIR USE IN TREATMENT OF CANCER

(71) Applicant: THE JOHNS HOPKINS UNIVERSITY, Baltimore, MD (US)

(72) Inventor: Alan D. Friedman, Baltimore, MD (US)

(73) Assignee: THE JOHNS HOPKINS UNIVERSITY, Baltimore, MD (US)

(21) Appl. No.: 17/106,893

(22) Filed: Nov. 30, 2020

Related U.S. Application Data
(63) Continuation of application No. PCT/US2019/033670, filed on May 23, 2019.
(60) Provisional application No. 62/677,815, filed on May 30, 2018.

(51) Int. Cl. A61K 35/28; A61P 35/00; A61K 45/06; C12N 5/0789

(52) U.S. Cl. CPC A61K 35/28; A61P 35/00; C12N 15/102; C12N 5/0647; A61K 45/06

(57) ABSTRACT

The present invention provides methods for making autologous bone marrow hematopoietic progenitors lacking NF-κB p50 protein subunit (p50). The progenitor cells are expanded, exposed to a myeloid cytokine, and provided intravenously to treat various malignancies. The infused cells have the potential to generate mature granulocytes, monocytes, macrophages, and dendritic cells that are activated due to the absence of p50. Methods for the genetically manipulation of a subject's hematopoietic progenitors during the expansion phase to reduce or eliminate p50 expression are also contemplated, and these progenitor cells may be combined with other therapeutic agents to maximize efficacy.

### REFERENCE TO RELATED APPLICATIONS

This application is a continuation of PCT Application No. PCT/2019/033670, filed May 23, 2019, which claims the benefit of U.S. Provisional Patent Application No. 62/677,815, filed on May 30, 2018, both of which are hereby incorporated by reference for all purposes as if fully set forth herein.

### STATEMENT OF GOVERNMENTAL INTEREST

This invention was made with government support under grant no. W81XWH-16-1-0334 awarded by the ARMY/Medical Research and Materiel Command. The government has certain rights in the invention.

### BACKGROUND OF THE INVENTION

In the U.S. population, mortality associated with the 15 most common cancer types alone has been estimated to approach 170 deaths annually per 100,000 individuals. Currently, there are an estimated 1,437,180 new cases of cancer and 565,650 deaths each year. The economic burden of cancer has been estimated to exceed $96B in 1990 dollars.

The nuclear factor kappa-light-chain-enhancer of activated B cells (NF-κB) activates inflammatory pathways in myeloid cells in response to extra-cellular signals. The canonical NF-κB subunits are p65 and p50; both contain the Rel Homology Domain that mediates homo- or hetero-dimerization and DNA-binding, with p65 also having a trans-activation domain. NF-κB p50 (p50) is an inhibitory subunit; in the basal state p65 is held in the cytoplasm by IκB, whereas p50:p50 homo-dimers enter the nucleus, bind DNA, and repress gene expression. Absence of p50 leads to activation of pro-inflammatory pathways.¹⁻³

T cell checkpoint inhibition has emerged as a novel cancer therapeutic approach effective in a subset of cancer patients. Effectiveness of T cell checkpoint inhibition is often limited by T cell-suppressive tumor myeloid cells. Thus, there exists an urgent need to improve cancer immunotherapy. Development of novel approaches that augment T cell checkpoint therapy, or that augment other treatments designed to increase anti-tumor T cell immunity such as tumor vaccines, will lead to clinical benefit for a large number of cancer patients.

Clinical trials have been conducted in cancer patients using mature macrophages, typically generated from blood monocytes using GM-CSF, activated using IFNγ, and provided intravenously (IV); these trials were largely unsuccessful, with the exception of instillation of macrophages into the bladder of patients with bladder cancer, which proved less effective than standard therapy.^{4,5} When radio-labeled and provided to patients IV, macrophages were seen in lung, liver, and spleen, but not tumors, perhaps limiting their efficacy,⁴ inspiring the present inventor to consider infusion of immature myeloid cells (IMC) that might more effectively reach tumors.

Infusion of anti-inflammatory, M2-polarized macrophages into mice with autoimmune encephalitis reverses CNS lesions, and infusion of murine bone marrow-derived monocytes similarly polarized to an immunosuppressive state reduces graft versus host disease (GVHD).^{6,7} Infused monocytes are more effective against GVHD if they are genetically modified, by deletion of both copies of the ASC gene, to retain their anti-inflammatory state in vivo,⁷ inspiring the present inventor to consider the utility of infusing pro-inflammatory IMC having absent or reduced NF-κB p50 as therapy for cancer.

### SUMMARY OF THE INVENTION

The present inventor has now developed methods for expanding myeloid progenitors from p50-/- or WT mice and find that adoptive cell transfer (ACT) of p50-/- immature myeloid cells, given after a single dose of 5-fluorouracil (5FU), leads to tumor responses in three cancer models, glioblastoma, prostate carcinoma, and pancreatic ductal carcinoma.

In accordance with one or more embodiments, the present invention provides methods for making autologous bone marrow hematopoietic progenitors wherein the level of expression of the NF-κB p50 protein subunit of said progenitor cell or cells is absent or reduced when compared to wild-type cells.

The bone marrow hematopoietic progenitor cells of the present invention are then expanded, exposed to a myeloid cytokine or cytokines, and can be used to treat various malignancies. The infused cells, designated p50-immature myeloid cells (p50-IMC), have the potential to generate mature granulocytes, monocytes, macrophages, and dendritic cells in vivo that are activated due to the absence of p50. Methods for the genetically manipulation of a subject's hematopoietic progenitors during the expansion phase to reduce or eliminate p50 expression are also contemplated, and the use of the inventive progenitor cells can be combined with chemotherapy or other therapeutic agents to maximize efficacy.

In some embodiments, the therapeutic agents that can augment p50-IMC anti-cancer efficacy include but are not limited to radiation therapy, T cell checkpoint inhibitors, epigenetic modulators, CSF1R antibodies or chemical inhibitors, metabolic modulators, tumor or dendritic cell vaccines, and myeloid cytokines.

In accordance with an embodiment, the present invention provides a synthetic hematopoietic progenitor cell or population of cells, wherein the NF-κB p50 protein subunit of said cell or cells is absent or reduced.

In accordance with an embodiment, the present invention provides a pharmaceutical composition comprising a synthetic hematopoietic progenitor cell or population of cells, wherein the expression level of the NF-κB p50 protein subunit of said cell or cells is absent or reduced when compared to wild-type, and a pharmaceutically acceptable carrier.

In accordance with an embodiment, the present invention provides a pharmaceutical composition comprising a synthetic hematopoietic progenitor cell or population of cells, wherein the expression of the NF-κB p50 protein subunit of said cell or cells is absent or reduced when compared to wild-type, a pharmaceutically acceptable carrier, and at least one or more additional biologically active agents.

In accordance with an embodiment, the present invention provides a method of treating cancer in a subject in need thereof, comprising administering to the subject a therapeutically effective amount of said cells or said pharmaceutical compositions described herein.

In accordance with an embodiment, the present invention provides a method for making a synthetic hematopoietic progenitor cell or population of cells, wherein the NF-κB p50 protein subunit of said cell or cells is absent or reduced, comprising obtaining bone marrow or blood cells from a mammal, isolating a population that includes hematopoietic stem and progenitor cells (e.g. isolation of lineage-negative or CD34+ cells), expanding these cells in vitro in the presence of FL, TPO, and SCF, and potentially additional or alternative cytokine combinations or other biologically activate agents that maintain cell immaturity, while introducing reagents designed to knockout (KO) their p50 genes or knockdown (KD) their p50 mRNAs encoding p50 protein, and to then culture the cells with M-CSF and/or other myeloid cytokines that might include GM-CSF, IL-4, and FL to generate p50-IMC.

### BRIEF DESCRIPTION OF THE DRAWINGS

FIGS. 1A-1B illustrate an embodiment of a method for making a synthetic hematopoietic immature myeloid cell population, wherein the expression of the NF-κB p50 protein subunit of said cell or cells is absent or reduced when compared to wild-type. The bone marrow from a p50-/- knockout mouse is flushed from the extremity bones and subjected to red blood cell lysis using ammonium chloride. Lineage-negative cells are isolated using a magnetic column after staining with a cocktail of antibodies that bind mature blood cells. These cells are then expanded for 6 days in IMDM media with heat-inactivated fetal bovine serum (HI-FBS) and the FL, TPO, and SCF cytokines, with penicillin/streptomycin (P/S). These cytokines maintain cell immaturity, leading to a blast-like morphology upon Wright-Giemsa staining, as shown for cells expanded from the marrow of a p50-/- mouse (1A, left). The cells are then transferred to IMDM with HI-FBS, P/S, and M-CSF for 1 day in ultra-low attachment plates, inducing an immature myeloid morphology in the majority of the cells (1A, right). At this point ˜87% of the cells express than pan-myeloid CD11b surface marker upon flow cytometry (FC), with ˜57% of CD11b+ cells expressing the monocyte and macrophage marker MCSFR and ˜22% instead expressing the dendritic cell (DC) marker FLT3 and CD11c (1B). 6% of the cells express the Ly6G granulocyte marker and none express the CD3 T cell or CD19 B cell markers (not shown). Cells obtained using the same protocol using WT marrow have similar morphologic and FACS characteristics.

FIGS. 1C-1E evaluate the in vivo localization of IMC. Wild-type (WT)-IMC cells were generated from CMV-Luc mice harboring a luciferase transgene. When a single dose of WT-IMC cells was infused into PDC, glioma, or PCa tumor-bearing mice, 5 days after a dose of 5-fluorouracil (5FU), a subset of cells reach the tumor, with some cells also localizing to lung, spleen, and bone marrow (1C). Additional experiments utilize the CD45.1/CD45.2 congenic allele pair. CD45.1 B6 mice implanted with pancreatic ductal carcinoma (PDC) received 5FU followed by two infusions of 1E7 CD45.2+ p50-IMC on day 12 (d12) and d14. Analysis on d16 shows that 8% of CD11b+ tumor myeloid cells derived from p50-IMC (1D). WT CD45.1 mice inoculated with prostate carcinoma (PCa) cells received 5FU on d13 followed by a single dose of CD45.2+ p50-IMC on d18. 2% of tumor on d2, and 13% of bone marrow (BM), 24% of spleen, and 1.4% of inguinal node CD11b+ myeloid cells on d6 derived from CD45.2+ p50-IMC (1E).

FIG. 1F evaluates the nature of myeloid cells derived from p50-IMC in vivo. The majority of PCa tumor CD45.2⁺CD11b⁺ cells express F4/80, CD11c, and MHCII, indicative of activated macrophages and/or DCs, whereas few tumor CD45.2+CD11b+ cells express the granulocytic Ly6G marker (1F, top), and a similar pattern is seen within the tumor-draining inguinal nodes (not shown). In contrast, p50-IMC-derived marrow cells express variable F4/80 and little CD11c or the MHCII activation marker, with a prominent Ly6G+ subset, indicative of donor-derived macrophages and granulocytes (1F, bottom), and a similar pattern was evident in spleen (not shown). Thus, p50-IMC form activated tumor and lymph node myeloid cells, with marrow and spleen potentially providing an ongoing reservoir.

FIGS. 2A-2C evaluate the effects of p50-IMC on tumor T cells. WT CD45.1+ mice inoculated with Hi-Myc PCa cells received 5FU on day 13 followed by 1×10⁷ CD45.2+ WT-IMC (n=5) or p50-IMC (n=4) on days 18, 21, and 25. Six days later, tumor cells were analyzed for CD3+CD4+ and CD3+CD8+ T cells within the CD45.2+ gate (2A), showing that p50-IMC increase tumor CD8 T cell ˜5-fold. Tumor CD4 and CD8 T cells were analyzed for intracellular IFNγ, 4 hours after exposure to vehicle or PMA/ionomycin, with brefeldin A/monensin protein transport inhibitors, showing that p50-IMC increase IFNγ+ activated tumor T cells ˜2-fold (2B). Tumor CD8 T cells were analyzed for PD-1 (2C), showing ˜2-fold increased T cell PD-1 after p50-IMC, supporting the potential utility of combining p50-IMC with anti-PD-1 T cell checkpoint inhibitors.

FIGS. 3A-3C demonstrate anti-cancer efficacy of p50-IMC. Ten mice inoculated SQ with Hi-Myc B6 prostate cancer cells^{8,9} showed significantly slower tumor growth after receiving 5FU (150 mg/kg) on day 13 followed by 1×10⁷ p50-IMC on days 18, 21, and 25, compared to 10 mice receiving WT myeloid cells or 5 mice receiving 5-fluorouracil (5FU) alone (3A). Mice implanted in the brain with GL261-Luc glioblastoma (GBM) cells^{10,11} received 5FU on day 3, followed by no myeloid cells, p50-IMC or WT-IMC on days 8, 11, and 14, and In Vivo Imaging System (IVIS) imaging on d21 (3B). While the 5FU or 5FU+WT-IMC groups had mice with large tumors or mice that died of tumors (indicated by XX), 3 of 5 mice subjected to p50-IMC ACT developed very small GBM tumors, with one having a large tumor and one having died prior to d21 (X). Finally, 4 of 10 mice inoculated in the pancreas with PDC-Luc cells showed marked regression in tumor size in response to 5FU+p50-IMC, but not 5FU+WT-IMC or 5FU alone (3C)—data in the left panel were obtained by providing 5FU on day 7, followed by p50-IMC on days 12, 15, and 19; data in the right panel were obtained by providing 5FU on day 3, following by WT-IMC or p50-IMC cells on days 7, 10, and 12 (note that tumor size is on a log scale). PDC-Luc cells were generated by introducing the Luc2 luciferase cDNA was into the UN-KC-6141 line.¹² 5FU is given to reduce marrow competition with infused IMC and to reduce tumor myeloid cell numbers.¹³

FIGS. 4A-4C demonstrate CRISPR/Cas9 knockout (KO) of the p50 gene in murine or human myeloid cell lines and shRNA knockdown (KD) of p50 mRNA in murine marrow myeloid progenitors, illustrative of alternative approaches that can be taken to develop human p50-IMC from patient hematopoietic cells for clinical application wherein the NF-κB p50 protein subunit of said cell or cells is absent or reduced. M1 murine or U937 human myeloid cell lines were transduced with lentiCRISPRv2 plasmids encoding either a non-targeting sgRNA (NTV), mouse or human p50 gene-directed sgRNAs, hSpCas9, and puromycin-resistance. After selection in puromycin for 7 d, pooled M1 or U937 cells were subjected to Western blotting for p50 and β-actin. Relative expression of p50 protein, normalized to β-actin, is shown below each lane (4A). PCR amplification of a fragment surrounding the Cas9 cut site of targeted and control cells followed by DNA sequencing and analysis with TIDE software evaluates p50 alleles;¹⁴ TIDE analysis of M1 lines sg2, sg3, and sg5 shows 78-93% allele targeting (not shown), with U937 sg1 cells having 89% p50 gene KO (4B). Murine BM, isolated 5 d after 5FU, were cultured in IMDM/FBS with SCF/FL/TPO and transduced with pLKO.1 lentiviral vectors expressing a non-targeting shRNA (NTV) or p50-directed shRNAs. After puromycin selection and transfer to M-CSF for 1 d, total cellular proteins were evaluated for p50 and β-actin (4C).

FIGS. 5A-5D. (5A) Experimental design: Hi-Myc murine prostate cancer (PCa) was inoculated SQ on d0, followed by 5FU on d13 and either no IMC, WT-IMC, or p50-IMC on days 18, 21, and 25. (5B) Tumor volumes for the individual mice in each group. (5C) Tumor volumes fit to an exponential model, with 95% confidence intervals. P-value was calculated for 5FU alone vs 5FU/p50-IMC on d30 in the context of the exponential model using the Student's t test with correction for multiple comparisons using tukey's method. (5D) Separate from the exponential model, tumor volumes as measured on d28 or d29 (left) and on d36 (right) were compared using the Student's t test. There are fewer mice on d36 due to the need to euthanize mice once tumors reach 2 cm in maximum linear dimension.

FIGS. 6A-6D. (6A) Experimental design: Murine pancreatic ductal carcinoma (PDC) cells expressing luiciferase were inoculated orthotopically on d0, followed by 5FU on d3 (Exp. 1) or d7 (Exps. 2 and 3), and either no IMC, WT-IMC, or p50-IMC on days 12, 15, and 19 (Exp. 1) or days 8, 11, and 14 (Exps. 2 and 3). (6B) Relative tumor bioluminescence (log scale), indicative of tumor size, for each tumor on different days in each mouse. (6C) Tumor volumes for Exps. 2 and 3 fit to an exponential model, with 95% confidence intervals. P-Value as calculated for 5FU/WT-IMC vs 5FU/p50-IMC on d14 in the context of the exponential model using the Student's t test. (6D) Separate from the exponential model, tumor volumes as measured on d14 from Experiements. 2 and 3 and were compared using the Student's t test.

### DETAILED DESCRIPTION OF THE INVENTION

In accordance with an embodiment, the present invention provides a synthetic hematopoietic progenitor cell or population of cells, wherein the expression of the NF-κB p50 protein subunit of said cell or cells is absent or reduced when compared to wild-type.

In some embodiments, the present invention provides compositions, methods, and systems for generating synthetic hematopoietic progenitor cell or population of cells, wherein the expression of the NF-κB p50 protein subunit of said cell or cells is absent or reduced when compared to wild-type.

In some embodiments, the present invention provides compositions, systems, and methods for administering synthetic hematopoietic progenitor cell or population of cells, wherein the expression of the NF-κB p50 protein subunit of said cell or cells is absent or reduced when compared to wild-type to a subject (e.g., to a subject with cancer in an adoptive transfer type of procedure).

In some embodiments, these cells of the present invention, where the expression of the NF-κB p50 protein subunit of said cell or cells is absent or reduced when compared to wild-type are termed immature myeloid cells (IMC) or “p50-IMC”.

In some embodiments, p50-IMC are generated from mammals such as mice lacking both copies of the gene encoding p50. In some alternative embodiments, p50-IMC are generated from hematopoietic cells, such as those obtained from a cancer patient, using gene editing tools such as CRISPR/Cas9 to knockout (KO) one or both p50 gene alleles in a subset of the cells. In some embodiments, p50-IMC are generated from hematopoietic cells, such as those obtained from a cancer patient, using agents that knockdown (KD) expression of p50 mRNA, such as shRNA, siRNA, anti-sense DNA, or anti-sense RNA. In some embodiments, p50-IMC express the monocyte markers CD11b, MCSFR, CD14, CD64, and/or CD16. In some embodiments, IMC express the dendritic cell markers HLA-DR, CD209, and/or FLT3. In other embodiments, IMC can also express CD11c, CD1c, CD141, CD303, CD304, CD1a, CD15, CD13, and/or CD33.

In accordance with an embodiment, the present invention provides a pharmaceutical composition comprising a synthetic hematopoietic progenitor cell or population of cells, wherein the expression of the NF-κB p50 protein subunit of said cell or cells is absent or reduced when compared to wild-type, and a pharmaceutically acceptable carrier.

In accordance with an embodiment, the present invention provides a pharmaceutical composition comprising a synthetic hematopoietic progenitor cell or population of cells, wherein the expression of the NF-κB p50 protein subunit of said cell or cells is absent or reduced when compared to wild-type, a pharmaceutically acceptable carrier, and at least one or more additional biologically active agents.

In accordance with an embodiment, the present invention provides a method of treating cancer in a subject in need thereof, comprising administering to the subject a therapeutically effective amount of said cells or said pharmaceutical compositions described herein.

In accordance with an additional embodiment, the present inventions provides a method for making a synthetic hematopoietic progenitor cell or population of cells, wherein the expression of the NF-κB p50 protein subunit of said cell or cells is absent or reduced when compared to wild-type, comprising blood cells developed from mammalian induced pluripotent stem cells (iPSC). In this embodiment, reagents to knockout (KO) or knockdown (KD) the p50 mRNAs encoding p50 proteins are introduced into iPSC before culturing under conditions optimized to generate blood cells (for example as in ref. 22), followed by isolating a population that includes hematopoietic stem and progenitor cells (e.g., isolation of lineage-negative or CD34+ cells). These cells can then be expanded in vitro, in the presence of Flt3 ligand (FL), thrombopoietin (TPO), and stem cell factor (SCF), and potentially additional or alternative cytokine combinations, or other biologically active agents that maintain cell immaturity. The cells can be further cultured with M-CSF and/or other myeloid cytokines that can include GM-CSF, IL-4, and FL, to generate p50-IMC.

As an additional embodiment, hematopoietic stem and progenitor cells would be isolated from iPSC after culture under conditions optimized to generate blood cells, with p50 KO or KD as these cells are expanded in conditions that maintain their immaturity, prior to transfer to myeloid cytokines to generate p50-IMC.

As used herein, the term “wherein the expression of the NF-κB p50 protein subunit of said cell or cells is absent or reduced when compared to wild-type” means that the p50 protein subunit is not expressed in the cell or population of cells at detectable levels, or the level of expression of the p50 protein subunit in the cell or population of cells is less than the level of expression of a control or a wild-type cell or population of cells.

As used herein, the term “regression” refers to the return of a diseased subject, cell, tissue, or organ to a non-pathological, or less pathological state as compared to basal nonpathogenic exemplary subject, cell, tissue, or organ. For example, regression of a tumor includes a reduction of tumor mass as well as complete disappearance of a tumor or tumors.

As used herein the term, “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reactions that occur within a natural environment.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

In some embodiments, the p50-IMC can be further expanded in vitro by exposure to IL-3, IL-6, Notch ligands or aryl hydrocarbon antagonists.^{15,16}

In some embodiments, the inventive methods include when a patient receives immunotherapy with one or more checkpoint inhibitors, prior to, at the same time, and/or after receiving the p50-IMC by adoptive transfer IV, or prior to, at the same time, and/or after direct administration of p50-IMC to the patient's tumor. In various embodiments, the checkpoint inhibitor(s) target one or more of CTLA-4 or PD-1/PD-L1, and/or other checkpoint inhibitors such as LAG-3 or TIM-3, which may include antibodies against such targets, such as monoclonal antibodies, or portions thereof, or humanized or fully human versions thereof. In some embodiments, the checkpoint inhibitor therapy comprises Yervoy (ipilimumab) or Keytruda (pembrolizumab).

In some embodiments, the inventive methods include when the patient receives a tumor or dendritic cell vaccine prior to, at the same time, and/or after receiving the p50-IMC by adoptive transfer IV, or prior to, at the same time, and/or after direct administration of p50-IMC to the patient's tumor. In various embodiments, the tumor vaccine might be autologous tumor cells expressing GM-CSF or tumor cells mixed with other cells expressing GM-CSF. In some embodiments, the dendritic cell vaccine may be patient dendritic cells primed with a tumor antigen.

In some embodiments, the inventive methods include when the patient receives radiation therapy to the tumor prior to or subsequent to receiving p50-IMC by adoptive transfer IV or prior to or subsequent to direct administration of p50-IMC to the patient's tumor.

In some embodiments, the inventive methods include when the patient receives about 1 to 5 rounds of adoptive immunotherapy (e.g., one, two, three, four or five rounds) with the p50-IMC. In some embodiments, each administration of adoptive immunotherapy is conducted prior to (e.g., from about 1 day to about 1 week prior to), simultaneously with, or after (e.g., from about 1 day to about 1 week after), a round of checkpoint inhibitor therapy.

In particular embodiments, the inventive methods further comprise administering the synthetic hematopoietic progenitor cell or population of cells, the p50-IMC to a subject (e.g. a patient). In some embodiments, the subject has a tumor and the contacting reduces the size (or eliminates) the tumor.

The subject referred to in the inventive methods can be any host. Preferably, the host is a mammal. As used herein, the term “mammal” refers to any mammal, including, but not limited to, mammals of the order Rodentia, such as mice and hamsters, and mammals of the order Lagomorpha, such as rabbits. It is preferred that the mammals are from the order Carnivora, including Felines (cats) and Canines (dogs). It is more preferred that the mammals are from the order Artiodactyla, including Bovine (cows) and Swine (pigs) or of the order Perssodactyla, including Equine (horses). It is most preferred that the mammals are of the order Primates, Ceboids, or Simoids (monkeys) or of the order Anthropoids (humans and apes). An especially preferred mammal is the human.

In some embodiments, the inventive methods further comprise administering to the subject cytokines active on myeloid or T cells that might include M-CSF, G-CSF, GM-CSF, FL, IL-4, IL-2, and/or IL-15, or chemical mimetics mimicking the action of one or more of these cytokines. In certain embodiments, after a subject has been treated with the adoptive transfer methods and compositions of the present invention, diagnostic procedures are employed to determine efficacy. In certain embodiments, tumor regression is analyzed. For example, clinical and radiographic responses (e.g. MRI and CT) can be used for monitoring the effector tumor-reactive p50-IMC on tumor growth. Certain procedures include clinical, histological and bioluminescent in vivo imaging for monitoring tumor growth. In some embodiments, the persistence of functional p50-IMC is monitored by isolation of myeloid cells from tumor or draining lymph node followed by analysis for p50 mRNA or protein expression or p50 gene deletion or by staining tumor or lymph node tissue for proteins or RNAs expressed in activated myeloid cells, including in macrophages and dendritic cells. In other embodiments, the ability of p50-IMC to induce an anti-tumor T cell response is monitored by staining tumor or lymph node tissue for total and activated CD4 and CD8 T cells and for regulatory T cells or by isolation of tumor or lymph node T cell followed FC or RNA isolation and mRNA analysis.

Administration and Dosing Regimes.

One skilled in the art will appreciate that administration and dosing of cells for adoptive transfer may need to be customized to the patient for highest efficacy and tolerance. In human patients, this translates to a dose of about 3×10⁸ p50-IMC cells, although higher and lower amounts of cells (e.g. one or more orders of magnitude different) may be employed. It is noted that, in certain embodiments, the number of p50-IMC cells that is needed for therapeutic treatment using the methods and compositions of the present invention is generally less than disclosed in the prior art. For example, in some embodiments, the amount of the p50-IMC applied to the patient can be e.g., 3×10⁷ to 1×10⁸ cells. It is further noted that while repeated transplantation can improve the efficacy of p50-IMC-mediated anti-tumor activity, embodiments of the present invention may employ one or more administrations of the p50-IMC. Such therapy may be sufficient for therapeutic treatment and may be further augmented by repeated checkpoint inhibitor, tumor or dendritic cell vaccine, and cytokine therapy.

Types of Cancer.

Methods of some embodiments of the present invention find use in the treatment of cancer and are not limited by the type of cancer. In some embodiments, methods may be directed towards treatment of solid tumors. Examples of solid tumors include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. Additional types of malignancies and related disorders include but are not limited to leukemia (acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia, chronic lymphocytic leukemia), polycythemia vera, lymphoma (Hodgkin's disease, non-Hodgkin's disease), multiple myeloma, Waldenström's macroglobulinemia, melanoma, sarcoma, and heavy chain disease.

It will be understood by those of ordinary skill in the art that the term “tumor” as used herein means a neoplastic growth which may, or may not be malignant. Additionally, the compositions and methods provided herein are not only useful in the treatment of tumors, but in their micrometastses and their macrometastses. Typically, micrometastasis is a form of metastasis (the spread of a cancer from its original location to other sites in the body) in which the newly formed tumors are identified only by histologic examination; micrometastases are detectable by neither physical exam nor imaging techniques. In contrast, macrometastses are usually large secondary tumors.

Co-Administration With Chemotherapeutic Agents.

Chemotherapy and the adoptive p50-IMC cell transfer of the present invention may be performed sequentially or simultaneously. For example, myeloid depleting chemotherapy may be conducted prior to adoptive cell transfer. The present invention is not limited by type of anti-cancer agent co-administered. Indeed, a variety of anti-cancer agents are contemplated to be useful in the present invention including, but not limited to, Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carubicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N42-(Dimethyl-amino)ethyllacridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Fluorocitabine; Fosquidone; Fostriecin Sodium; FK-317; FK-973; FR-66979; FR-900482; Gemcitabine; Geimcitabine Hydrochloride; Gemtuzumab Ozogamicin; Gold Au 198; Goserelin Acetate; Guanacone; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N,N′-Bis(2-chloroethyl)-N-nitrosourea (BCNU); N-(2-chloroethyl)-N′-cyclohex-yl-N-nitrosourea (CCNU); N-(2-chloroethyl)-N′-(trans-4-methylcyclohexyl-N-nitrosourea (MeCCNU); N-(2-chloroethyl)-N′-(diethyl)ethylphosphonate-N-nit-rosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis (platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); and 2-chlorodeoxyadenosine (2-Cda).

With respect to the pharmaceutical compositions used in combination with the p50-IMC described herein, the carrier can be any of those conventionally used for cell therapy, and is limited only by considerations such as cell viability and by the route of administration. The carriers described herein are well-known to those skilled in the art and are readily available to the public. It is preferred that the carrier be one which is chemically inert to the active agent(s), and one which has little or no detrimental side effects or toxicity under the conditions of use. Examples of the carriers include tissue culture media or buffered saline, and these carriers may include cytokines used to generate p50-IMC.

The choice of carrier will be determined, in part, by the particular pharmaceutical composition, as well as by the particular method used to administer the composition. Accordingly, there are a variety of suitable formulations of the pharmaceutical composition of the invention.

It will be understood to those of skill in the art that the term “chemotherapeutic agent” is any agent capable of affecting the structure or function of the body of a subject or is an agent useful for the treatment or modulation of a disease or condition in a subject suffering therefrom. Examples of therapeutic agents can include any drugs known in the art for treatment of disease indications, including, for example, cancer.

An active agent and a biologically active agent are used interchangeably herein to refer to a chemical or biological compound, including cells that induce a desired pharmacological and/or physiological effect, wherein the effect may be prophylactic or therapeutic.

The dose of the chemotherapeutic agents used in conjunction with the p50-IMC of the present invention also will be determined by the existence, nature and extent of any adverse side effects that might accompany the administration of a particular composition. Typically, an attending physician will decide the dosage of the pharmaceutical composition with which to treat each individual subject, taking into consideration a variety of factors, such as age, body weight, general health, diet, sex, compound to be administered, route of administration, and the severity of the condition being treated. By way of example, and not intending to limit the invention, the dose of one or more chemotherapeutic agents used in conjunction with p50-IMC can be about 0.001 to about 1000 mg/kg body weight of the subject being treated, from about 0.01 to about 100 mg/kg body weight, from about 0.1 mg/kg to about 10 mg/kg, and from about 0.5 mg to about 5 mg/kg body weight.

The dose of the compositions of the present invention also will be determined by the existence, nature and extent of any adverse side effects that might accompany the administration of a particular composition. Typically, an attending physician will decide the dosage of the pharmaceutical composition with which to treat each individual subject, taking into consideration a variety of factors, such as age, body weight, general health, diet, sex, compound to be administered, route of administration, and the severity of the condition being treated.

As used herein, the terms “effective amount” or “sufficient amount” are equivalent phrases which refer to the amount of a therapy (e.g., a prophylactic or therapeutic agent), which is sufficient to reduce the severity and/or duration of a disease, ameliorate one or more symptoms thereof, prevent the advancement of a disease or cause regression of a disease, or which is sufficient to result in the prevention of the development, recurrence, onset, or progression of a disease or one or more symptoms thereof, or enhance or improve the prophylactic and/or therapeutic effect(s) of another therapy (e.g., another therapeutic agent) useful for treating a disease, such as a neoplastic disease or tumor.

As noted above, compositions comprising the p50-IMC of the invention can be administered parenterally by injection, infusion or implantation (subcutaneous, intravenous, intratumor, intraperitoneal, or the like) in dosage forms, formulations, or via suitable delivery devices or implants containing conventional, non-toxic pharmaceutically acceptable carriers and adjuvants.

In a preferred embodiment, the dosage form of the p50-IMC is suitable for injection or intravenous administration.

Exemplary Methods for Making the p50-IMC of the Present Invention.

1) p50-IMC From p50-/- Mice.

Bone marrow is flushed from the extremity bones and subjected to red blood cell lysis using ammonium chloride. Lineage-negative (Lin−) cells are isolated using a magnetic column, a chemical affinity column, or flow cytometry sorting, after staining with a cocktail of antibodies (e.g. targeting CD3, B220, CD11b, Gr-1, and Ter119) that bind mature blood cells. Alternatively, CD34+ cells are isolated by staining with CD34 antibody followed by affinity column chromatography or flow cytometry. The Lin− or CD34+ cells are then expanded for 6-16 days in IMDM media with 10% heat-inactivated fetal bovine serum (HI-FBS) and the FL (30 ng/mL), TPO (10 ng/mL), and SCF (30 ng/mL) cytokines, with 1× penicillin-streptomycin (P/S). These cytokines maintain cell immaturity. The cells are then transferred to IMDM with 10% HI-FBS, 1×P/S, and M-CSF (10 ng/mL) or GM-CSF (10 ng/mL) or GM-CSF (10 ng/mL) with IL-4 (10-40 ng/mL) for 1-2 days in ultra-low attachment plates, inducing formation of immature myeloid cells.

2) p50-IMC From Human Bone Marrow.

For clinical application, CRISPR/Cas9 can be used to knockout (KO) the p50 gene to generate human p50-IMC. In addition, p50 shRNA can be used to knockdown (KD) the p50 RNA to generate human p50-IMC. CD34+ hematopoietic stem and progenitor cells would be isolated from patient bone marrow or peripheral blood, for example using nanobead-conjugated CD34 antibody and immunomagnetic selection.¹⁷ These cells will then be expanded for 6-16 days (and potentially longer), preferably in serum-free medium such as X-Vivo-20 with FL (30-100 ng/mL), TPO (10-100 ng/mL), and SCF (30-100 ng/mL) cytokines, potentially under hypoxic (e.g. 5% oxygen) conditions, and potentially in the presence of additional biologic agents. Lentiviral vectors (LV) expressing either Cas9/sgRNA (for KO) or shRNA (for KD) designed to target human p50 will be packaged in 293T cells using pMDLg/pRRE, pRSV-Rev, and pMD2.G, or related LV packaging plasmids, followed by concentration of cell supernatants. CD34+ marrow cells will then transduced for 2-3 days as they expand using purified LV with Retronectin-coated plates, via spinoculation, or in liquid culture, potentially in the presence of 4-8 μg/mL protamine sulfate.¹⁸ As a second method for p50 gene KO, Cas9 protein can be combined with HPLC-purified 100 bp sgRNA having 2′-O-methyl and phosphorothioate stabilizing modifications on three 5′ and 3′ nucleotides, e.g. in a 1:2.5 molar ratio, to generate ribonucleoprotein complexes (RNPs), followed by nucleofection into CD34+ cells as they expand.¹⁹ Additional methods for p50 gene KO include co-nucleofection of chemically stabilized sgRNA and Cas9 mRNA or nucleofection of plasmids encoding Cas9 and p50 sgRNAs.^{19,20} Use of two sgRNAs targeting different segments of the p50 gene might be used in each of these methods of p50 gene KO. After p50 gene KO or KD and cell expansion, the cells will be transferred to serum-free media with myeloid cytokines such as M-CSF (10-100 ng/mL), GM-CSF (10-100 ng/mL), or GM-CSF (10-100 ng/mL) with IL-4 (10-100 ng/mL) or FL (10-100 ng/mL) for 1-3 days prior to cell infusion. We are currently utilizing p50 gene KO, using sgRNAs cloned into LentiCRISPRv2 LV, and p50 mRNA KD, and using shRNAs in pLKO.1 LV, in the M1 murine and human U937 myeloid cell lines and in murine marrow cells expanding in TPO/FL/SCF.

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### EXAMPLES

Generation of Murine WT-IMC or p50-IMC.

The inventive progenitor cell products are generated as follows: Bone marrow from WT or p50-/- mice is flushed from the extremity bones and subjected to red blood cell lysis using ammonium chloride. Lineage-negative cells are isolated using a magnetic column after staining with a cocktail of biotin-anti-Lineage antibodies (CD3, B220, CD11b, Gr-1, and Ter119) that bind mature blood cells, and anti-biotin microbeads. The obtained Lin− cells are then expanded for 6 days in IMDM media with 10% heat-inactivated fetal bovine serum (HI-FBS) and the 30 ng/mL FL, 10 ng/mL TPO, and 30 ng/mL SCF cytokines, with 1× penicillin-streptomycin (P/S). The cells are then transferred to IMDM with 10% HI-FBS, P/S, and 10 ng/mL M-CSF for 1 day in ultra-low attachment plates. Cell morphology is assessed by Wright's-Giemsa staining of cell cytospins. Cell surface marker expression is assessed by flow cytometry (FC) using anti-CD45-BV650, anti-FLT3-BV421, anti-MCSFR-PE, and anti-CD11c-PE-Dazzle (Biolegend) and anti-CD11b-PerCPCy5.5 (BD) antibodies.

GBM Cell Inoculation and Tumor Monitoring.

GL261-Luc cells are grown in DMEM with 10% FBS. Mice are anesthetized with intraperitoneal (IP) ketamine/xylazine; the dorsal neck is shaved and sterilized and a 1 cm sagittal cranial incision is made to expose the skull; a burr hole is drilled 0.1 mm anterior and 2.25 mm lateral to the bregma; a 10 μL syringe with a 27 g needle is inserted to 4 mm and 1.3×10⁵ GL261-Luc cells injected over 15 min; the incision is closed with a staple and adhesive. Glioblastoma tumor growth is monitored by IVIS imaging.

Prostate Cancer Cell Inoculation and Tumor Monitoring.

When SQ Hi-Myc prostate cancer (PCa) tumors reached 1.2-1.5 cm, mice are euthanized and tumor tissue collected, minced with a razor blade, washed with phosphate-buffered saline (PBS), resuspended in 5 mL DMEM/F12 media containing 10% FBS, 500 μL Collagenase/Hyaluronidase (Stem Cell Technologies), 2.5 U/mL Dispase and 0.05 mg/mL DNase I, and then incubated at 37° C. for 1 hr with occasional mixing. Tumor tissue is passed through a 40 μM cell strainer with the aid of a syringe plunger. Cells are then pelleted at 350 g×5 min and resuspended in phosphate-buffered saline (PBS). Live cells are enumerated using Trypan Blue dye and a hemocytometer, and 2×10⁶ viable cells in 100 μL PBS are injected SQ into the shaved flank of mice anesthetized with isofluorane. Tumor growth is monitored using caliper measurements of length (L), width (W) and height (H), with volume estimated from the ellipsoid volume formula: V=L×W×H×π/6.

Pancreatic Cancer Cell Inoculation and Tumor Monitoring.

PDC-Luc cells are grown in DMEM with 10% FBS. Mice anesthetized with ketamine/xylazine are subjected to hair removal and disinfection of the operative site with Providone Iodine/Alcohol. A 1 cm incision is made in the left flank using a sterile scalpel and a 5 mm incision in the peritoneum. The entire pancreatic body together with spleen is pulled out and exposed to the outside of the peritoneal cavity by using a pair of blunt-nose forceps. 0.5×10⁶ PDC-Luc cells in 40 μL Matrigel is injected using a 30 g needle and a Hamilton syringe into the tail of the pancreas. The pancreas is then be returned to the peritoneal cavity with blunt forceps. The muscle layer and skin are closed separately with 4-0 absorbable sutures. Pancreatic cancer tumor growth is monitored by IVIS imaging.

Treatment of Tumor-Bearing Mice With 5FU Alone or With IMC.

5FU is given IP at 150 mg/kg. Tail vein infusions of PBS, 1×10⁷ WT-IMC, or 1×10⁷ p50-IMC are given starting 5 d after the dose of 5FU and then every 2-4 days for three total doses. Tumor growth and murine survival is then monitored.

Tumor Myeloid and T Cell Isolation.

Prostate cancer or pancreatic cancer tumors are dissociated using 500 μL Collagenase/Hyaluronidase (Stem Cell Technologies), 2.5 U/mL Dispase and 0.05 mg/ml DNase I, and subjected to FC analysis, gating on live cells lacking staining with Live/Dead Aqua (ThermoFischer).

Tumor, Node, Marrow, and Spleen Myeloid and T Cell Subset and Activation.

Spleen and lymph node cells are dissociated by passage through 40 μM cell strainers. All antibody staining is preceded by 15 min of 1:50 FcγR block in FC buffer, on ice. Extracellular antibodies are then added for 45 min on ice. Intracellular staining is accomplished after surface staining using the Foxp3 staining kit (eBioscience). Myeloid subsets are stained with anti-CD11b-FITC, anti-CD45-BV650, anti-Ly6C-AF700, anti-MR-PE-Cy7, anti-CD11c-PE/Dazzle594, anti-Ly6G-BV605 (BioLegend), anti-MHCII-eFluor450 (eBioscience), and anti-F4/80-APC (BioRad). To assess T cell activation, total tumor cells are incubated for 4 hr at 37° C. in a 5% CO₂ incubator with Protein Transport Inhibitor Cocktail containing brefeldin A and monensin (eBioscience). Cells are then stained with anti-CD3-AF488, anti-CD4-PE, and anti-CD8-PerCP-Cy5.5 followed by intracellular stain with anti-IFNγ-APC (BioLegend). Anti-CD45.1-PE-Cy7 and anti-CD45.2-BV650 antibodies are employed to distinguish CD45.2+ IMC from CD45.1+ host cells.

Western Blot Analyses.

Total cellular proteins prepared in Laemmli sample buffer are subjected to Western blotting using p50 (13586, Cell Signaling) and β-actin (AC-15, Sigma) antibodies.

Data Analysis.

Prostate tumor growth is analyzed using Multiple Comparisons of Means: Tukey Contrasts. T cell subsets are compared using the Student t test.

Animal Source.

WT C57BL/6 (B6) mice are obtained from Charles River Laboratories, Nfkb1(p50)-/- and CMV-Luc mice are from Jackson Laboratory (stock # s 6097, 25854).

Cell Lines.

Hi-Myc PCa cells were developed from a metastatic prostate cancer lesion that formed in B6 mice expressing c-Myc from the probasin promoter.⁹

GL261-Luc cells were obtained from Perkin Elmer. UN-KC-6141 PDC cells¹² were provided by Dr. Surinder K. Batra at the University of Nebraska Medical Center. These were stably transfected with the Luc2 cDNA (Promega), with G418 selection followed by isolation of subclones by limiting dilution and screening to identify one with high-level luciferase activity, designated as PDC-Luc cells.

Retroviral Transduction.

DNA oligonucleotides encoding sgRNAs targeting the murine or human p50 gene were inserted into the LentiCRISPRv2 LV vector, which were then transfected into 293T cells with the pCMV-ΔR8.91 and pMD.G(VSV.G) LV packaging plasmids using Lipofectamine 2000, followed by collection of cell supernatant 2 d and 3 d later to obtain LV particles. These were then filtered through 0.45 μM low protein-binding filters and used to transduce M1 or U937 myeloid cells in RPMI with 10% HI-FBS in the presence of 4 μg/mL Polybrene. Cells were then cultured in the presence of 2 μg/mL puromycin to select for transduced cells. pLKO.1 LV vectors expressing shRNAs targeting p50 mRNA were packaged similarly and used to transduce Lin− murine marrow isolated from WT mice in media containing IMDM, 10% HI-FBS, 10 ng/mL TPO, 30 ng/mL FL, and 30 ng/mL SCF in the presence of 4 μg/mL Polybrene.

### Example 1

Generation of p50-IMC and Their Localization and Myeloid Potential.

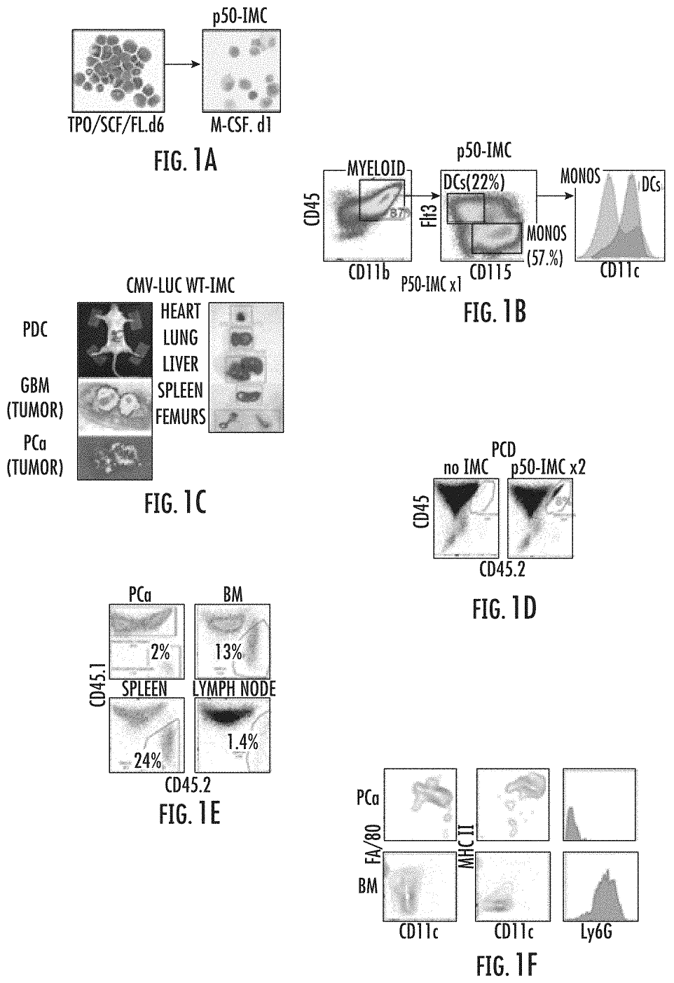

Lin⁻ BM cells from p50-/- mice were expanded for 6 d in TPO/SCF/FL in IMDM/FBS and the transferred to M-CSF in the same media for 1 d to generate p50-IMC. Morphology after Wright-Giemsa staining shows blast morphology in TPO/SCF/FL and immature monocytic morphology in M-CSF (FIG. 1A). p50-IMC were subjected to FC, demonstrating that the majority of cells express the pan-hematopoietic surface marker CD45 and the pan-myeloid surface marker CD11b, and that amongst these myeloid cells ˜22% are Flt3+CD115-CD11c+ DCs and ˜57% are Flt3-CD115+CD11c− monocytes (FIG. 1B). WT-IMC were generated from CMV-Luc mice using the same protocol and infused (5 d after a dose of 5-FU 150 mg/kg IP) into mice inoculated previously with PDC, GBM, or PCa cells (lacking Luc). Mice or cut tumors and isolated organs were imaged by IVIS 2 d later, demonstrating localization of infused IMC to the tumors, as well as to lung, spleen, and bone marrow (FIG. 1C). CD45.1 mice inoculated with PDC received 5FU on d7 and CD45.2+ p50-IMC on d12 and d14, followed by FC for CD11b, CD45, and CD45.2 on d16, demonstrating that at that time ˜8% of tumor myeloid cells derived from p50-IMC (FIG. 1D). Mice inoculated with PCa received 5FU on d13 and p50-IMC on d18. Myeloid cell subsets were than analyzed by FC in tumor on d2 and in spleen, draining nodes, and BM, on d6, demonstrating that about 2% of tumor, 13% of marrow, 24% of spleen, and 1.4% of lymph node myeloid cells derived from p50-IMC (FIG. 1E). Tumor and marrow cells were subjected to FC for F4/80;CD11c, MHCII;CD11c, or Ly6G within the CD45.2+CD11b+ myeloid subset of PCa tumor cells two days or BM cells six days after one p50-IMC infusion, demonstrating that within the tumor p50-IMC generated F4/80+ macrophages that express CD11c and MHCII, signs of an activated state (FIG. 1F).

### Example 2

Effects of p50-IMC on Tumor T Cell Activation and PD-1 Expression.

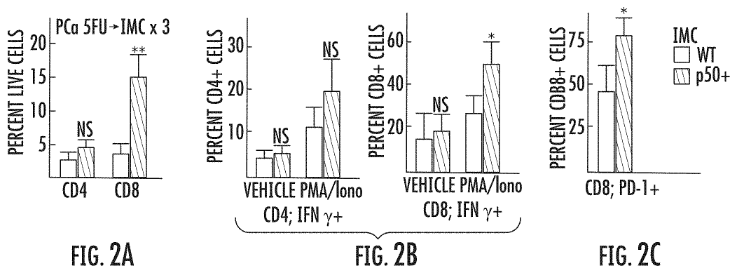

To evaluate the effect of p50-IMC on T cell activation CD45.1+ mice inoculated with Hi-Myc PCa received 5FU on d13 followed by CD45.2+ WT-IMC or p50-IMC on days 18, 21, and 25, followed by isolation of tumor and inguinal nodes six days later. Total tumor CD8 T cells were increased 5-fold by p50-IMC (FIG. 2A), with ˜2-fold increase in IFNγ+, activated CD8 T cells evident after 4 hr stimulation with PMA/ionomycin (FIG. 2B). Similarly, 7.7% of lymph node CD8 T cells expressed IFNγ after WT-IMC compared with 14.7% after p50-IMC, in response to PMA/ionomycin (p=0.004), and 1.8% of lymph node CD4 T cells were IFNγ+ after WT-IMC compared with 5.3% after p50-IMC (p=0.007, not shown). In addition, the percent of tumor CD8 T cells expressing PD-1 increased almost 2-fold after p50-IMC (FIG. 2C), supporting the potential utility of combining p50-IMC with anti-PD-1 T cell checkpoint inhibitor antibody.

### Example 3

Efficacy of p50-IMC Against Murine Prostate Cancer.

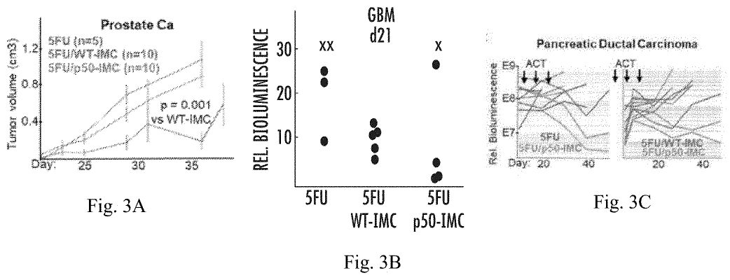

The efficacy of 5FU, 5FU+p50-IMC, and 5FU+WT-IMC were compared. Cells were given vial tail vein injection, 1×10⁷ cells/dose, 2-4 days apart, starting 5 days after a single dose of 5FU, as diagrammed (FIG. 3A). 5FU is given to reduce marrow competition with infused IMC, to reduce tumor myeloid cell numbers, and potentially release tumor neoantigens to augment immune response. Fourteen mice inoculated SQ with Hi-Myc PCa cells showed significantly slower tumor growth after receiving 5FU on day 13, followed by p50-IMC on days 18, 21, and 25, when compared to 13 mice receiving WT myeloid cells or 18 mice receiving 5FU alone (FIG. 3B). These data were fitted to an exponential model of tumor growth, with tumor volumes plotted on a log-scale; statistical analysis shows highly significant slowing of tumor growth comparing 5FU versus 5FU/p50-IMC on multiple days, including on day 30 as indicated, and tumor growth after 5FU/WT-IMC was even faster than with 5FU alone (FIG. 3C). Independent of the exponential tumor growth, tumor volumes as measured on days 28 or 29 were 3-fold smaller after 5FU/p50-IMC versus 5FU alone, and they were 4-fold smaller on day 36, with slightly higher p values (FIG. 3D).

These data show that a single dose of 5FU followed by three doses of p50-IMC is highly effective at slowing murine prostate cancer growth compared with 5FU alone or 5FU followed by three doses of WT-IMC.

### Example 4

Efficacy of p50-IMC Against Murine Pancreatic Ductal carcinoma.

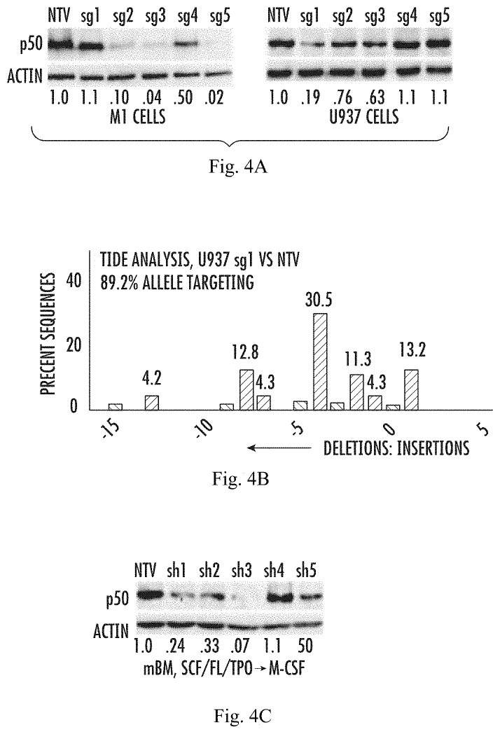

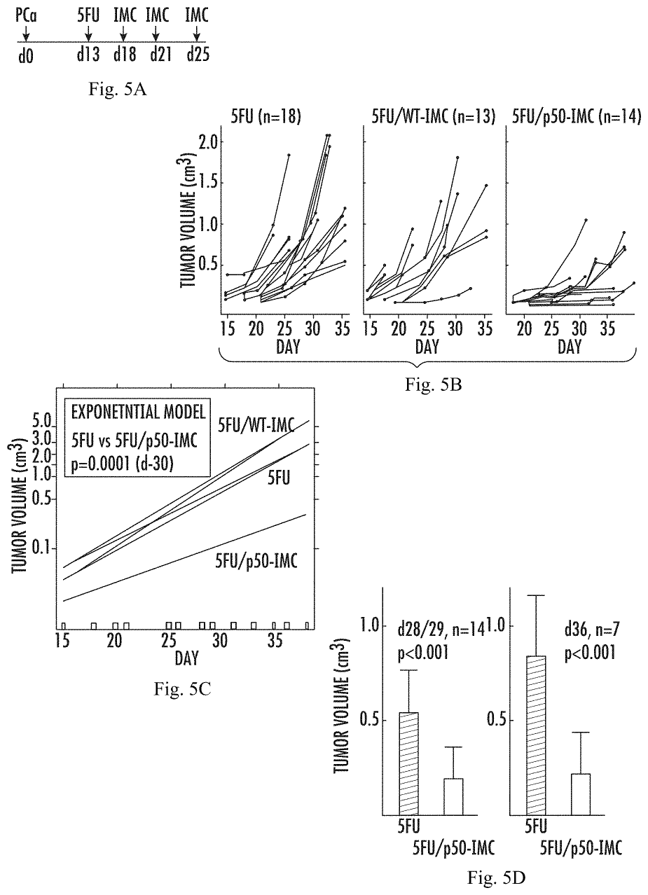

The efficacy of 5FU, 5FU+p50-IMC, and 5FU+WT-IMC were compared. Cells were given vial tail vein injection, 1×10⁷ cells/dose, 3-4 days apart, starting five days after a single dose of 5FU, with 5FU given either on day 7 (Experiment 1) or day 3 (Experiments 2 and 3), as diagrammed (FIG. 4A). 6 of 15 mice inoculated orthotopically with PDC-Luc cells showed marked regression in tumor size, as assessed by tumor bioluminescence, in response to 5FU/p50-IMC, compared with 5FU/WT-IMC or 5FU alone (FIG. 4B)—note that tumor size is on a log scale. Experiment 2 and 3 data were fitted to an exponential model of tumor growth; statistical analysis shows highly significant slowing of tumor growth comparing 5FU/p50-IMC versus 5FU/WT-IMC on day 14, as indicated (FIG. 4C). Independent of the exponential tumor growth model, tumor volumes as measured on day 14 were 3-fold smaller after 5FU/p50-IMC versus 5FU/WT-IMC alone, with a highly significant p values (FIG. 4D).

These data show that the a single dose of 5FU followed by three doses of p50-IMC is highly effective at slowing murine pancreatic cancer growth compared with 5FU alone or 5FU followed by three doses of WT-IMC.

### Example 5

Efficacy of p50-IMC Against Murine Glioblastoma.

The efficacy of 5FU, 5FU+p50-IMC, and 5FU+WT-IMC were compared. Cells were given vial tail vein injection, 1×10⁷ cells/dose, 2-4 days apart, starting 5 days after a single dose of 5FU. Mice implanted orthotopically with GL261-Luc cells received 5FU on day 3, followed by no myeloid cells, p50-IMC or WT-IMC on days 8, 11, and 14, and IVIS imaging on d21. While the 5FU or 5FU+WT-IMC groups had mice with large tumors or mice that died of tumors (indicated by X), 3 of 5 mice subjected to p50-IMC ACT developed very small GBM tumors, with one having a large tumor and one having died prior to d21 (FIG. 3B).

These data show that single dose of 5FU followed by three doses of p50-IMC is highly effective at slowing murine glioblastoma cancer growth compared with 5FU alone or 5FU followed by three doses of WT-IMC.

### Example 6

p50 Gene Knockout (KO) and mRNA Knockdown (KD) in Murine and Human Myeloid Cell Lines and in Murine Bone Marrow Cells.

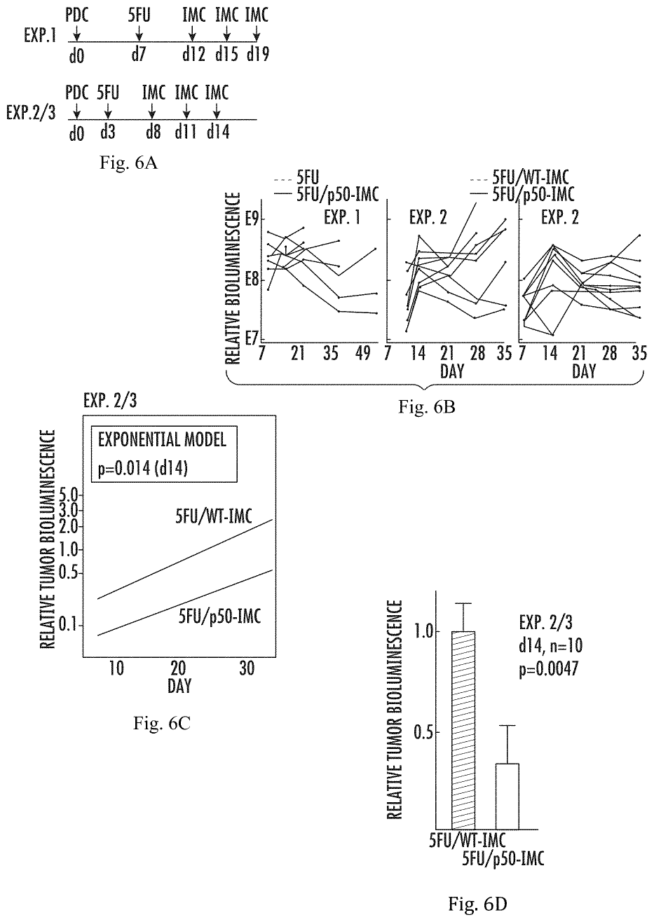

We designed five murine and human p50 exon sgRNAs using a Broad Institute website²¹ and introduced the corresponding oligonucleotides into the lentiCRISPRv2 plasmid, which encodes the sgRNA, hSpCas9, and puromycin-resistance. p50 protein is markedly reduced in several pooled M1 or U937 transductants after puromycin selection, suggesting biallelic KO in the majority of clones (FIG. 6A). PCR amplification of a fragment surrounding the Cas9 cut site of targeted and control cells followed by DNA sequencing and analysis with TIDE software evaluates p50 alleles;¹⁴ TIDE analysis of M1 lines sg2, sg3, and sg5 shows 78-93% allele KO (not shown), with U937 sg1 cells having 89.2% p50 gene KO due to DNA insertions and deletions (FIG. 6B). In addition, we have identified p50 shRNAs effective in reducing p50 protein in stably transduced, puromycin-selected murine marrow myeloid progenitors (FIG. 6C).

Without being held to any particular theory, it is thought that the immaturity of p50-IMC may facilitate tumor localization and also allow DC formation, which might each contribute to efficacy. While there is previously published data showing slower melanoma, fibrosarcoma, and colon cancer growth in p50-/- mice, however, these mice lack p50 in all cell types, and the present inventors demonstrate for the first time efficacy of p50-IMC ACT. Whether generation of p50-IMC cells is best done in humans using TPO/FL/SCF followed by M-CSF for 1 day or via a related protocol (e.g. using GM-CSF instead of M-CSF) and whether reducing or eliminating p50 protein expression during expansion in TPO/FL/SCF is best done using CRISPR/Cas9 or shRNA LV transduction, CRISPR/Cas9 protein, RNA, or plasmid nucleofection, or alternative approaches will need to be determined, as will assessment of what agents best synergize with human p50-IMC, e.g. 5FU, Cytoxan, MCSFR antibody, DNA methyltransferase inhibitors, checkpoint inhibitors, and/or tumor vaccines in each cancer type.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. Ghosh S, May M J, Kopp E B. NF-κB and Rel proteins: evolutionarily conserved mediators of immune responses. Annu. Rev. Immunol. 1998; 16:225-60.
2. Cartwright T, Perkins N D, L Wilson C. NFKB1: a suppressor of inflammation, ageing and cancer. FEBS J. 2016; 283(10):1812-22.
3. Porta C, Rimoldi M, Raes G, Brys L, Ghezzi P, Di Liberto D, Dieli F, Ghisletti S, Natoli G, De Baetselier P, Mantovani A, Sica A. Tolerance and M2 (alternative) macrophage polarization are related processes orchestrated by p50 nuclear factor κB. Proc. Natl. Acad. Sci. USA. 2009; 106(35):14978-83.
4. Lee S, Kivimae S, Dolor A, Szoka F C. Macrophage-based cell therapies: the long and winding road. J. Controlled Release 2016; 240:527-40.
5. Burger M, Thiounn N, Denzinger S, et al. The application of adjuvant autologous antravesical macrophage cell therapy vs. BCG in non-muscle invasive bladder cancer: a multicenter, randomized trial. J. Transl. Med. 2010; 8:54.
6. Weber M S, Prod'homme T, Youssef S, Dunn S E, Rundle C D, Lee L, Patarroyo J C, Stüve O, Sobel R A, Steinman L, Zamvil S S. Type II monocytes modulate T cell-mediated central nervous system autoimmune disease. Nat. Med. 2007; 13(8):935-43.
7. Koehn B H, Apostolova P, Haverkamp J M, Miller J S, McCullar V, Tolar J, Munn D H, Murphy W J, Brickey W J, Serody J S, Gabrilovich D I, Bronte V, Murray P J, Ting J P, Zeiser R, Blazar B R. GVHD-associated, inflammasome-mediated loss of function in adoptively transferred myeloid-derived suppressor cells. Blood 2015; 126(13):1621-8.
8. Ellwood-Yen K, Graeber T G, Wongvipat J, Irueala-Arispe M L, Zhang J, Matusik R, Thomas G V, Sawyers C L. Myc-driven murine prostate cancer shares molecular features with human prostate tumors. Cancer Cell 2003; 4(3):223-38.
9. Barakat D J, Suresh R, Barberi T, Pienta K, Simons B W, Friedman A D. Absence of myeloid Klf4 reduces prostate cancer growth with pro-atherosclerotic activation of tumor myeloid cells and infiltration of CD8 T cells. PLoS One 2018; 13(1):e0191188.
10. Seligman A M, Shear M J. Studies in carcinogenesis. VIII. Experimental production of brain tumors in mice with methylcholanthrene. Am. J. Cancer 1939; 37:364-95.
11. Maes W, Van Gool S W. Experimental immunotherapy for malignant glioma: lessons from two decades of research in the GL261 model. Cancer Immunol. Immunother. 2011; 60(2): 153-60.
12. Tones M P, Rachagani S, Souchek J J, Mallya K, Johansson S L, Batra S K. Novel pancreatic ductal carcinoma cancer cell lines derived from genetically engineered mouse models of spontaneous pancreatic adenocarcinoma: applications in diagnosis and therapy. PLoS One 2013:8(11):e8058016.
13. Vincent J, Mignot G, Chalmin F, Ladoire S, Bruchard M, Chevriaux A, Martin F, Apetoch L, Rebe C, Ghringhelli F. 5-fluorouracil selectively kills tumor-associated myeloid-derived suppressor cells resulting in enhanced T cell-dependent antitumor immunity. Cancer Res. 2010; 70(8):3052-61.
14. Brinkman E K, Chen T, Amendola M, van Steensel B. Easy quantitative assessment of genome editing by sequence trace decomposition. Nucl. Acids. Res. 2014; 42(22):e168.
15. Delaney C, Milano F, Othus M, Becker P S, Sandhu V, Nicoud I, Dahlberg A, Bernstein I D. Infusion of a non-HLA-matched ex-vivo expanded cord blood progenitor cell product after intensive acute myeloid leukemia chemotherapy: a phase 1 trial. Lancet Haematol. 2016; 3(7):e330-9.
16. Boitano A E, Wang J, Romeo R, Bouchez L C, Parker A E, Sutton S E, Walker J R, Flaveny C A, Perdew G H, Denison M S, Schultz P G, Cooke M P. Aryl hydrocarbon receptor antagonists promote the expansion of human hematopietic stem cells. Science 2010; 329(5997):1345-8.
17. Shpall E J, Gehling U, Cagnoni P, Purdy M, Hami L, Gee A P. Isolation of CD34-positive hematopoietic progenitor cells. Immunomethods 1994; 5:197-203.
18. Rio P, Navarro S, Guenechea G, Sanchez-Dominguez R, Lamana M L, Yanez R, Casado J A, Mehta P A, Pujol M R, Surralles J, Charrier S, Galy A, Segovia J C, Diaz de Heredia C, Sevilla J, Bueren J A. Engraftment and in vivo proliferation advantage of gene-corrected mobilized CD34+ cells from Fanconi anemia. Blood 2017; 130(13):1535-42.
19. Dever D P, Bak R O, Reinisch A, Camaraena J, Washington G, Nicolas C E, Pavel-Dinu M, Saxena N, Wilkens A B, Mantri S, Uchida N, Hendel A, Narla A, Majeti R, Weinberg K I, Porteus M H. CRISPR/Cas9 β-globin gene targeting in human haematopoietic stem cells. Nature 2016; 539(7629):384-9.
20. Mandal P K, Ferreira L M, Collins R, Meissner T B, Boutwell C L, Friesen M, Vrbanac V, Garrison B S, Stortchevio A, Bryder D, Musunuru K, Brand H, Tager A M, Allen T M, Talkowski M E, Rossi D J, Cowan C A. Efficient ablation of genes in human hematopoietic stem and effector cells using CRISPR/Cas9. Cell Stem Cell 2014; 15(5):643-52.
21. Doench J G, Fusi N, Sullender M, Hegde M, Vaimberg E W, Donovan K F, Smith I, Tothova Z, Wilen C, Orchard R, Virgin H W, Listgarten J, Root D E. Optimized sgRNA design to maximize activity and minimize off-target effects of CRISPR-Cas9. Nat. Biotech. 2016; 34(2): 184-95.
22. Salvagiotto G, Burton S, Daigh C A, Rajesh D, Slukvin I I, Seay N J. A defined, feeder-free, serum-free system to generate in vitro hematopoietic progenitors and differentiated blood cells from hESCs and hiPSCs. PLoS One 2011; 6(3): e17829.

1. A synthetic hematopoietic progenitor cell or population of cells, wherein expression of nuclear factor kappa-light-chain-enhancer of activated B cells (NF-κB) p50 protein subunit in said cell or population of cells is reduced when compared to wild-type cells.

2. The hematopoietic progenitor cell or population of cells of claim 1, wherein said cell or cells express one or more cell surface markers selected from the group consisting of: CD11b, CD115/MCSFR, CD14, CD64, CD16, HLA-DR, CD209, FLT3, CD11c, CD1c, CD141, CD303, CD304, CD1a, CD15, CD13, and CD33.

3. The hematopoietic progenitor cell or population of cells of claim 1, wherein said cell or population of cells are obtained from the bone marrow or blood of a mammal genetically modified to:
(a) lack at least one copy of the NF-κB p50 protein subunit gene, or
(b) to have reduced levels or activity of the mRNA for the NF-κB p50 protein subunit.

4. The hematopoietic progenitor cell or population of cells of claim 1, wherein said cell or population of cells are obtained from the bone marrow or blood of a mammal where the gene for the NF-κB p50 protein subunit was genetically deleted through the use of: a CRISPR/Cas9 gene editing construct, a zinc finger nuclease (ZFN), or a transcription activator-like effector nuclease (TALEN).

5. The hematopoietic progenitor cell or population of cells of claim 1, wherein said cell or population of cells are obtained from the bone marrow or blood of a mammal where the level or activity of the mRNA for the NF-κB p50 protein subunit is genetically reduced through the use of an shRNA, anti-sense RNA, or anti-sense DNA construct.

6. The hematopoietic progenitor cell or population of cells of claim 3, wherein the mammal is a human.

7. The hematopoietic progenitor cell or population of cells of claim 1, wherein said cell or population of cells are obtained from iPSC genetically modified to lack at least one copy of the p50 gene or to have reduced levels or activity of the p50 mRNA for the NF-κB p50 protein subunit.

8. The progenitor cell or population of cells of claim 7, wherein the iPSC cell or population of cells was genetically modified to lack both copies of p50 gene.

9. The hematopoietic progenitor cell or population of cells of claim 1, wherein said cell or population of cells are obtained by genetically modifying hematopoietic cells derived from iPSC to lack at least one copy of the p50 gene or to have reduced levels or activity of the p50 mRNA for the NF-κB p50 protein subunit.

10. The progenitor cell or population of cells of claim 9, wherein the population of cells was genetically modified to lack both copies of the p50 gene.

11. A pharmaceutical composition comprising the cell of claim 1 and a pharmaceutically acceptable carrier.

12. The pharmaceutical composition of claim 11, further comprising at least one additional therapeutic agent.

13. The pharmaceutical composition of claim 11 wherein the composition is in the form of a graft.

14. A method of treating a disease in a subject in need thereof, comprising administering to the subject a therapeutically effective amount of a synthetic hematopoietic progenitor cell or population of cells, wherein expression of NF-κB p50 protein subunit in said cell or population of cells is reduced when compared to wild-type cells; and wherein said disease is a cancer, non-cancerous aberrant cellular proliferation, or an infectious disease.

15. The method of claim 14, wherein the cancer is melanoma, sarcoma, colon carcinoma, pancreatic ductal carcinoma, glioblastoma, prostate carcinoma or neuroblastoma.

16. The method of claim 14, wherein the non-cancerous aberrant cellular proliferation is polycythemia vera.

17. The method of claim 14 wherein the infectious disease is caused by infection by a bacterium, virus, yeast, fungus, prion, protozoan, or helminth.

18. The method of claim 14, wherein the subject is first treated with 15-150 mg/kg 5-fluorouracil for 1-5 days and then the subject is administered 1×10⁵ to 5×10⁹ synthetic hematopoietic progenitor cells, wherein expression of NF-κB p50 protein subunit in said cells is reduced when compared to wild-type cells, every to 2 to 10 days later.

19. The method of claim 14, wherein the subject is first treated with a chemotherapy agent other than 5-fluoruracil for 1-5 days and then the subject is administered 1×10⁵ to 5×10⁹ synthetic hematopoietic progenitor cells, wherein expression of NF-κB p50 protein subunit in said cells is reduced when compared to wild-type cells, every to 2 to 10 days later.

20. The method of claim 14, wherein the subject also receives a T cell checkpoint inhibitor every 2-4 weeks targeting PD-1, PD-L1, PD-L2, CTLA-4, LAG-3, or TIM-3 beginning prior to, simultaneous to, and/or subsequent to 1×10⁵ to 5×10⁹ cells of synthetic hematopoietic progenitor cells, wherein expression of NF-κB p50 protein subunit in said cells is reduced when compared to wild-type cells, every to 2 to 10 days.

21. The method of claim 14, wherein the subject also receives a DNA methyltransferase inhibitor and/or a histone deacetylase inhibitor beginning prior to, simultaneous to, and/or subsequent to 1×10⁵ to 5×10⁹ cells of synthetic hematopoietic progenitor cells, wherein expression of NF-κB p50 protein subunit in said cells is reduced when compared to wild-type cells, every to 2 to 10 days.

22. The method of claim 14, wherein the subject also receives an inhibitor of CD47 or SIRPα beginning prior to, simultaneous to, and/or subsequent to 1×10⁵ to 5×10⁹ cells of synthetic hematopoietic progenitor cells, wherein expression of NF-κB p50 protein subunit in said cells is reduced when compared to wild-type cells, every to 2 to 10 days.

23. The method of claim 14, wherein the synthetic hematopoietic progenitor cells, wherein expression of NF-κB p50 protein subunit in said cells is reduced when compared to wild-type cells, and wherein the gene for SIRPα was genetically deleted through the use of: a CRISPR/Cas9 gene editing construct, a zinc finger nuclease (ZFN), or a transcription activator-like effector nuclease (TALEN).
